(19) United States
(12) Patent Application Publication
(10) Pub. No.: US 2023/0346317 A1
(43) Pub. Date: Nov. 2, 2023

(54) DETERMINATION APPARATUS

(71) Applicant: NEC Corporation, Minato-ku, Tokyo (JP)

(72) Inventors: Yuji OHNO, Tokyo (JP); Toshinori HOSOI, Tokyo (JP); Masahiro KUBO, Tokyo (JP)

(73) Assignee: NEC Corporation, Minato-ku, Tokyo (JP)

(21) Appl. No.: 18/025,776

(22) PCT Filed: Sep. 17, 2020
(86) PCT No.: PCT/JP2020/035296

§ 371 Date: Mar. 10, 2023

(51) Int. Cl. A61B 5/00; A61B 5/16

(52) U.S. Cl. CPC A61B 5/7221; A61B 5/74; A61B 5/165

(57) ABSTRACT

A determination apparatus (500) includes an estimation unit (512) configured to estimate a cause for an error based on data used when an agitation state is determined, and a notification unit (522) configured to issue a notification according to a result of the estimation by the estimation unit (521).

### TECHNICAL FIELD

The present invention relates to a determination apparatus, a notification method, and a recording medium.

### BACKGROUND ART

When a patient is in an agitated state, a risk of extubation, needle dislodgement, decannulation, tumble, a fall, and the like increases, and the patient may be, for example, injured as a result thereof. In this regard, there have been known techniques for determining a predictive sign of agitation to reduce such a risk and a possibility.

Examples of known documents discussing the techniques for determining a predictive sign of agitation include Patent Literature 1. Patent Literature 1 discusses a biological information processing system including a determination unit and an estimation unit. According to Patent Literature 1, the determination unit determines discrimination information indicating whether or not a condition of a patient has changed in comparison with a normal state based on features of biological information of the patient. Then, the estimation unit estimates countermeasure information for the patient based on the discrimination information determined by the determination unit and countermeasure prediction parameters learned in advance. Further, Patent Literature 1 discloses a heartbeat and the like as one example of the biological information, and discloses an agitation score indicating a possibility that the patient is in an agitated state as one example of the discrimination information.

### CITATION LIST

### Patent Literature

Patent Literature 1: International Publication No. 2019/073927

### SUMMARY OF INVENTION

### Technical Problem

In the case of the technique discussed in Patent Literature 1 or the like, occurrence of an error somewhere in the system makes it impossible to acquire the information required for the estimation. This may make it impossible to determine the agitation state as a result thereof.

The state that makes it impossible to determine the agitation state in the above-described manner is not a favorable condition, and therefore is desired to be solved immediately.

Under these circumstances, one of objects of the present invention is to provide a determination apparatus, a notification method, and a recording medium that allows an appropriate action to be taken when the agitation state cannot be determined for some reason.

### Solution to Problem

To achieve the above-described object, according to one aspect of the present disclosure, a determination apparatus is configured to include

an estimation unit configured to estimate a cause for an error based on data used when an agitation state is determined, and

a notification unit configured to issue a notification according to a result of the estimation by the estimation unit.

Further, according to another aspect of the present disclosure, a notification method is configured to cause a computer to

estimate a cause for an error based on data used when an agitation state is determined, and

issue a notification according to a result of the estimation.

Further, according to another aspect of the present disclosure, a recording medium records a program for causing a computer to realize processing including

estimating a cause for an error based on data used when an agitation state is determined, and

issuing a notification according to a result of the estimating.

### Advantageous Effects of Invention

According to each of the above-described configurations, the present invention allows an appropriate action to be taken when the agitation state cannot be determined for some reason.

### BRIEF DESCRIPTION OF DRAWINGS

FIG. 1 is a diagram illustrating an example of the overall configuration of an agitation determination system according to a first exemplary embodiment of the present disclosure.

FIG. 2 is a block diagram illustrating an example of the configuration of a sensor apparatus illustrated in FIG. 1.

FIG. 3 is a block diagram illustrating an example of the configuration of a bed terminal illustrated in FIG. 1.

FIG. 4 is a block diagram illustrating an example of the configuration of an agitation determination apparatus illustrated in FIG. 1.

FIG. 5 is a diagram illustrating one example of information stored in a storage unit.

FIG. 6 is a diagram illustrating one example of vital data included in sensing data.

FIG. 7 is a diagram illustrating one example of an agitation determination-use score included in score information.

FIG. 8 is a diagram for illustrating an example of processing by a score calculation unit.

FIG. 9 is a flowchart illustrating examples of operations of the agitation determination apparatus.

FIG. 10 is a block diagram illustrating another example of the configuration of the agitation determination apparatus.

FIG. 11 is a block diagram illustrating another example of the configuration of the agitation determination apparatus.

FIG. 12 is a diagram for illustrating one example of an error occurrence condition.

FIG. 13 is a diagram illustrating the hardware configuration of a determination apparatus according to a second exemplary embodiment of the present disclosure.

FIG. 14 is a block diagram illustrating an example of the configuration of the determination apparatus.

### DESCRIPTION OF EMBODIMENTS

### First Exemplary Embodiment

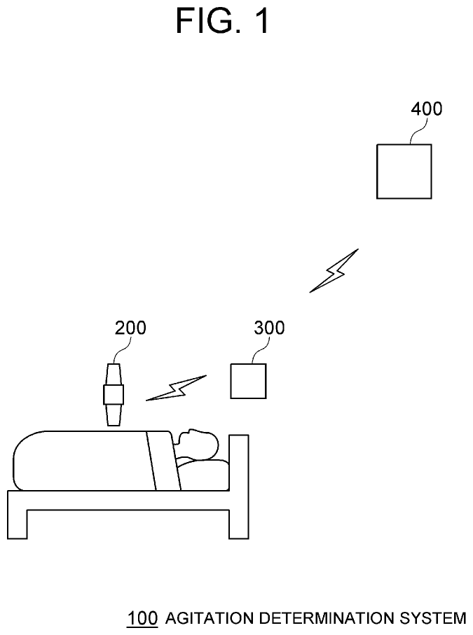

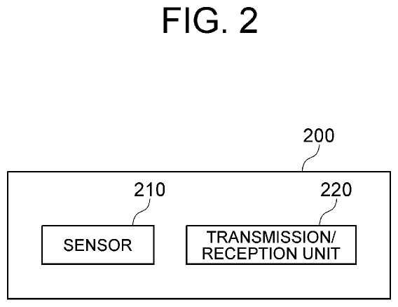

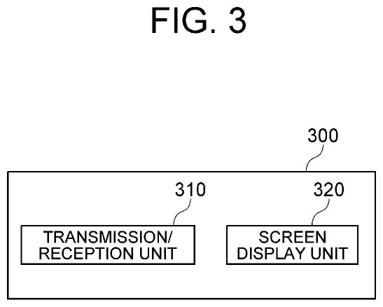

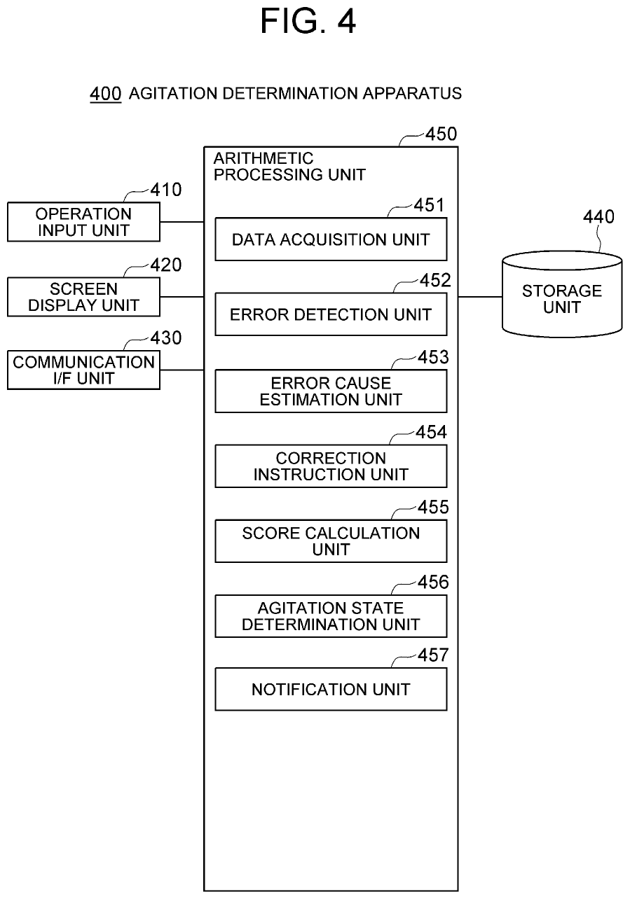

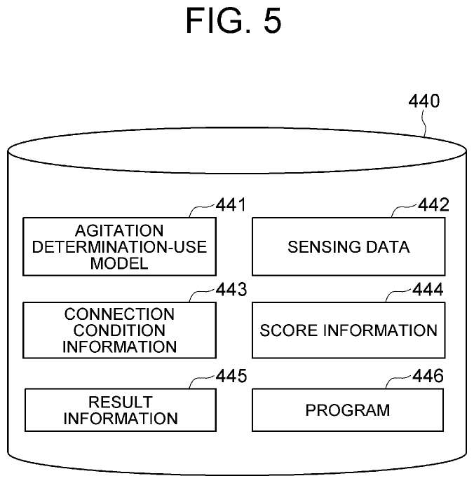

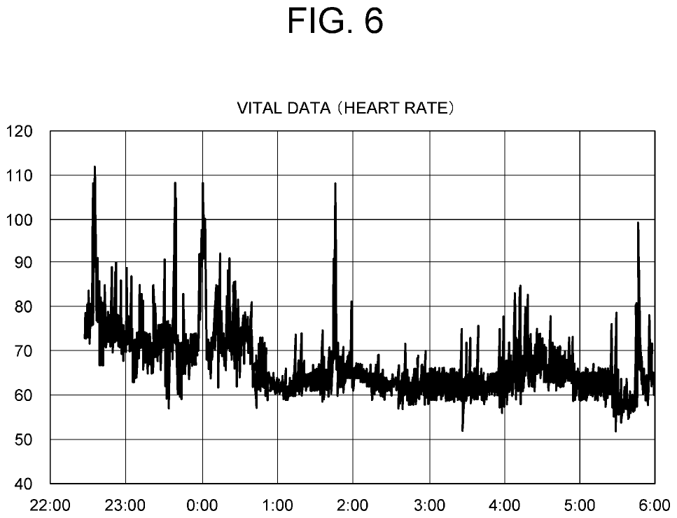

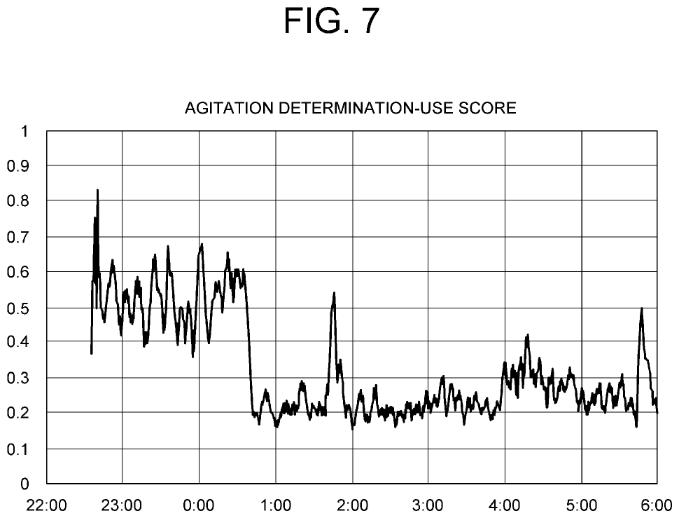

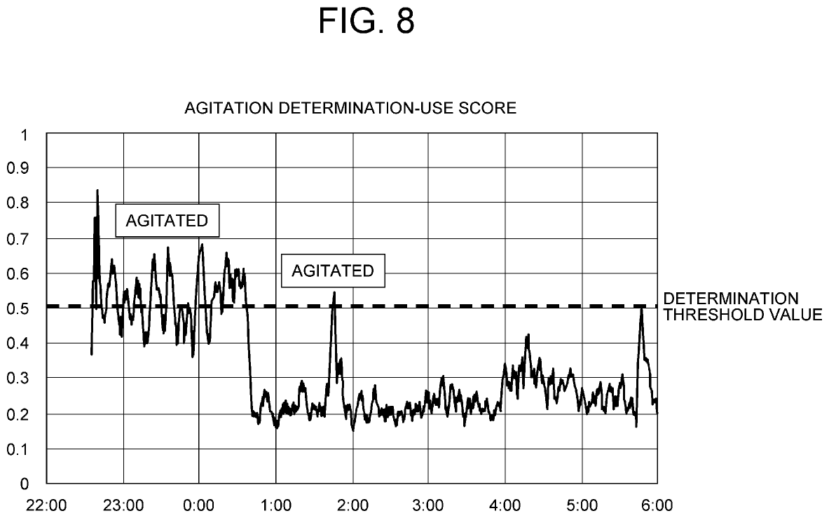

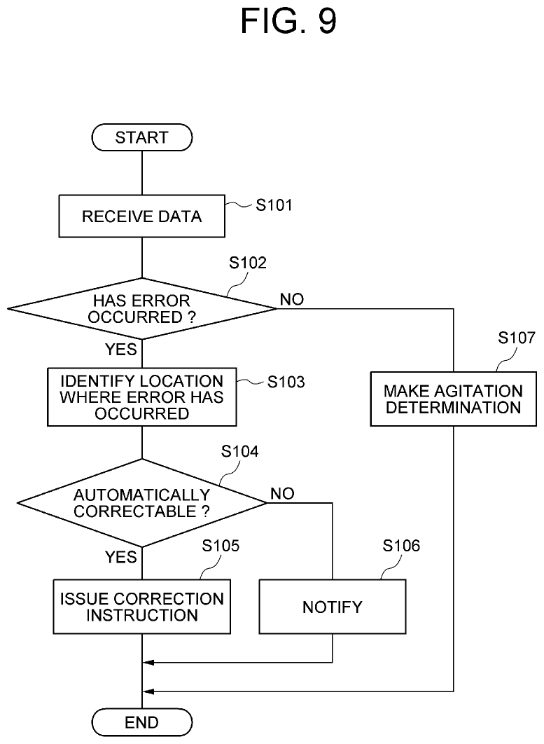

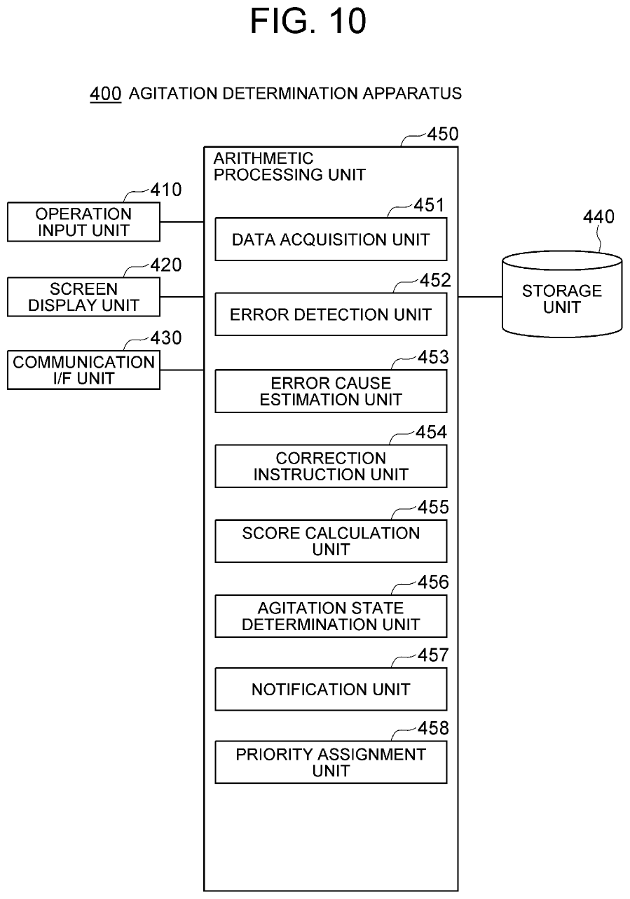

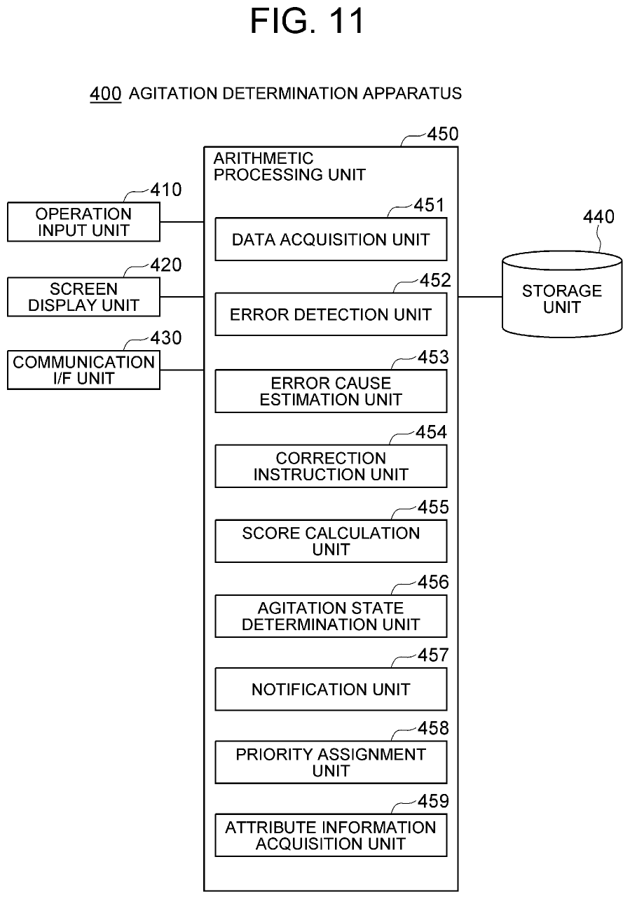

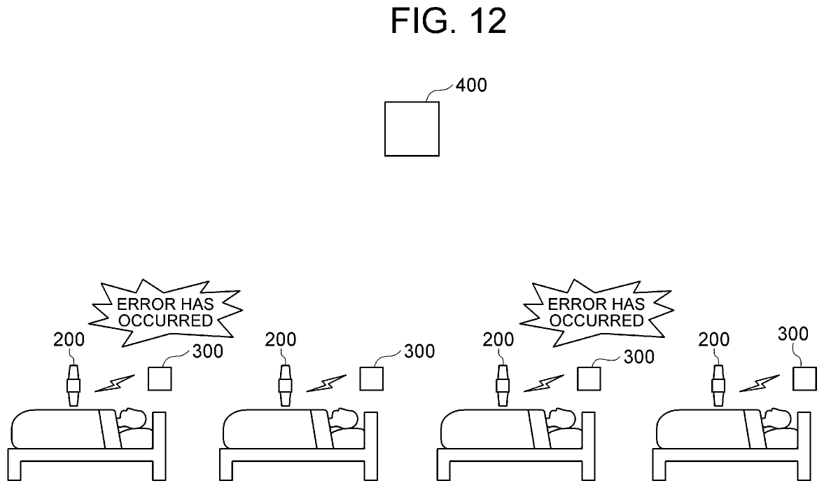

A first exemplary embodiment of the present disclosure will be described with reference to FIGS. 1 to 12. FIG. 1 is a diagram illustrating an example of the overall configuration of an agitation determination system 100. FIG. 2 is a block diagram illustrating an example of the configuration of a sensor apparatus 200. FIG. 3 is a block diagram illustrating an example of the configuration of a bed terminal 300. FIG. 4 is a block diagram illustrating an example of the configuration of an agitation determination apparatus 400. FIG. 5 is a diagram illustrating one example of information stored in a storage unit 440. FIG. 6 is a diagram illustrating one example of vital data included in sensing data 442. FIG. 7 is a diagram illustrating one example of an agitation determination-use score included in score information 444. FIG. 8 is a diagram for illustrating an example of processing by a score calculation unit 455. FIG. 9 is a flowchart illustrating examples of operations of the agitation determination apparatus 400. FIGS. 10 and 11 are block diagrams illustrating other examples of the configuration of the agitation determination apparatus 400. FIG. 12 is a diagram for illustrating one example of an error occurrence condition.

The first exemplary embodiment of the present disclosure will be described regarding the agitation determination system 100, which determines an agitation state of a patient based on data measured using the sensor apparatus 200. For example, the agitation determination system 100 calculates the agitation determination-use score based on the data measured by the sensor apparatus 200. Then, the agitation state determination system 100 determines the agitation state of the patient based on the calculated agitation determination-use score.

Further, as will be described below, the agitation determination system 100 detects an error that has occurred in the agitation determination system 100 and also identifies a location where the error has occurred according to, for example, a reception condition of the data in the agitation determination apparatus 400, which makes the agitation determination. Then, the agitation determination system 100 performs processing according to the result of the identification. For example, the agitation determination system 100 transmits a restart instruction for correcting the error to the identified location where the error has occurred and/or notifies an external terminal or the like that the error is identified. This allows an appropriate action to be taken even when the agitation determination system 100 cannot determine the agitation state for some reason.

Note that the agitation determination system 100, which will be described in the present exemplary embodiment, can be utilized in various scenes, such as an acute hospital, a convalescent hospital, a care facility, and monitoring at home. In the following description, the present exemplary embodiment will be described citing an example when the agitation determination system 100 is utilized in a hospital such as an acute hospital or a convalescent hospital. Note that the agitation determination system 100 may be utilized under a condition requiring the determination about the agitation state that is different from the above-described examples.

In the present exemplary embodiment, the agitation refers to a state in which the patient is restless and upset. The agitation may be caused by delirium or the like. Further, the agitation state indicates a state regarding the agitation of the patient. The agitation state indicates, for example, whether the patient is agitated or whether the patient exhibits a predictive sign of agitation. Note that the agitation state may include another index regarding a likelihood of the patient's agitation. When the patient is agitated, the patient may conduct a problematic behavior, such as falling off the bed, removing intubation, shouting abnormally, or committing violence. Therefore, it is desirable to accurately determine the agitation state.

FIG. 1 illustrates an example of the configuration of the agitation determination system 100. Referring to FIG. 1, the agitation determination system 100 includes, for example, the sensor apparatus 200, the bed terminal 300, and the agitation determination apparatus 400. As illustrated in FIG. 1, the sensor apparatus 200 and the bed terminal 300 are mutually communicably connected using short-range wireless communication such as Bluetooth (registered trademark), wired communication, or the like. Further, the bed terminal 300 and the agitation determination apparatus 400 are mutually communicably connected using short-range wireless communication such as Wi-Fi (registered trademark), wired communication, or the like. The bed terminal 300 and the agitation determination apparatus 400 may be connected via a relay apparatus such as a wireless base station.

Note that the number of sensor apparatuses 200, the number of bed terminals 300, and the number of agitation determination apparatuses 400 included in the agitation determination system 100 are not limited to the numbers exemplarily illustrated in FIG. 1. For example, the agitation determination system 100 can include a plurality of sensor apparatuses 200, a plurality of bed terminals 300, and/or a plurality of agitation determination apparatuses 400.

The sensor apparatus 200 measures, for example, vital data of the patient, which is a target. FIG. 2 illustrates an example of the configuration of the sensor apparatus 200. Referring to FIG. 2, the sensor apparatus 200 includes, for example, a sensor 210 and a transmission/reception unit 220. For example, the sensor apparatus 200 can realize each of the above-described processing units by hardware. The sensor apparatus 200 may realize each of the above-described processing units through execution of a program stored in a storage device by an arithmetic device such as a CPU.

The sensor 210 is a sensor that acquires the vital data and/or the like of the patient. As will be described below, time-series data that the sensor 210 acquires by measuring it can be used when, for example, the agitation of the patient is determined. For example, the sensor 210 is at least one of vital sensors such as a heart rate sensor, a respiratory rate sensor, a blood pressure sensor, a body temperature sensor, and a blood oxygen saturation level sensor.

Now, the vital data refers to a physical amount varying according to the vital activity of the patient. For example, the vital data includes at least one of a heart rate, a respiratory rate, a blood pressure value, a body temperature, a skin temperature, a blood flow rate, a blood oxygen saturation level, or the like of the patient.

Further, the sensor 210 can measure body motion data in addition to or instead of the vital data. For example, the sensor 210 can include at least one of body motion sensors such as an acceleration sensor, a gyro sensor (an angular velocity sensor), an angular sensor, and a microphone as a configuration for measuring the body motion data.

Note that the body motion data refers to a physical amount regarding the motion of the patient's body. For example, the body motion data includes at least one of an acceleration, an angular velocity, or an angle at a predetermined portion such as the patient's arm, body, or foot, a vocal volume, or the like.

The transmission/reception unit 220 includes an antenna or the like. The transmission/reception unit 220 transmits and receives data between the sensor apparatus 200 and the bed terminal 300.

For example, the transmission/reception unit 220 carries out communication using power-saving wireless communication such as Bluetooth between the sensor apparatus 200 and the bed terminal 300. For example, the transmission/reception unit 220 associates the vital data and/or the body motion data acquired by the sensor 210 with identification information of the sensor apparatus 200, and transmits them to the bed terminal 300.

This is an example of the configuration of the sensor apparatus 200. Note that the sensor apparatus 200 may be configured of one device or may be configured of a plurality of devices. For example, the sensor apparatus 200 may be realized by mutually communicably connecting a plurality of devices such as one or a plurality of sensor device(s) and a device having a function as the transmission/reception unit 230. Further, the sensor 210 may be worn by the patient or may be installed at the bed on which the patient stays or the like. Note that, in the case where the sensor apparatus 200 is configured of a plurality of devices, the plurality of devices may each include a transmission/reception unit.

The bed terminal 300 is an information processing apparatus set up in advance at, for example, a predetermined location such as around the bed on which the patient stays. The bed terminal 300 is, for example, a smartphone, and has a screen display function. The bed terminal 300 may be an apparatus different from the smartphone. Note that the bed terminal 300 is a terminal set up at a predetermined location where the patient should stay or a predetermined location based on which a range where the patient should stay is defined, and is not limited to the terminal set up around the bed.

FIG. 3 illustrates an example of the configuration of the bed terminal 300. Referring to FIG. 3, the bed terminal 300 includes, for example, a transmission/reception unit 310 and a screen display unit 320. For example, the bed terminal 300 can realize each of the above-described processing units by hardware. The bed terminal 300 may realize each of the above-described processing units through execution of a program stored in a storage device by an arithmetic device such as a CPU.

The transmission/reception unit 310 includes an antenna or the like, and transmits and receives data between the bed terminal 300, and the sensor apparatus 200 and the agitation determination apparatus 400. The transmission/reception unit 310 receives, for example, the body motion data and/or the vital data, and the identification information of the sensor apparatus 200 transmitted from the sensor apparatus 200. Then, the transmission/reception unit 310 transmits, for example, the body motion data and/or the vital data, and the identification information received from the sensor apparatus 200 to the agitation determination apparatus 400. Further, the transmission/reception unit 310 can receive information indicating a result of the determination about the agitation from the agitation determination apparatus 400.

Further, the transmission/reception unit 310 can transmit information according to a communication condition between the bed terminal 300 and the sensor apparatus 200 to the agitation determination apparatus 400. For example, if the power-saving wireless communication established between the bed terminal 300 and the sensor apparatus 200 is broken, the transmission/reception unit 310 can transmit information indicating that the communication between the bed terminal 300 and the sensor apparatus 200 is broken to the agitation determination apparatus 400.

The screen display unit 320 displays, for example, the vital data and/or the body motion data, and information indicating the result of the agitation determination and the state determination received by the transmission/reception unit 310 on a screen. For example, the screen display unit 320 can display that the patient corresponding to the bed terminal 300 is agitated or the like on the screen based on, for example, the received information indicating the result of the determination about the agitation.

The agitation determination apparatus 400 is an information processing apparatus that makes the agitation determination based on, for example, the vital data and/or the body motion data acquired by the sensor 210 included in the sensor apparatus 200. Further, the agitation determination apparatus 400 detects the occurrence of an error and also identifies a location where the error has occurred according to the state of the vital data and/or the body motion data, the reception condition thereof, and/or the like. The agitation determination apparatus 400 is set up at a predetermined location such as a nurse station. The agitation determination apparatus 400 is, for example, an information processing apparatus such as a personal computer or a tablet used by medical staff, a server set up in a hospital or the like, or a cloud server. The agitation determination apparatus 400 may be a combination of the information processing apparatus such as a personal computer or a tablet, and the server and/or the like. For example, the agitation determination apparatus 400 is a combination of an on-premise server in the hospital and a smartphone, and makes the determination based on the body motion data and/or the vital data by the on-premise server and displays the result and/or notifies the medical staff of the result on the smartphone used by the medical staff. Note that the medical staff is, for example, a doctor or a nurse. The medical staff according to the present disclosure is not limited thereto and may be any person who engages in a medical care.

FIG. 4 illustrates an example of the configuration of the agitation determination apparatus 400. Referring to FIG. 4, the agitation determination apparatus 400 includes, for example, an operation input unit 410, a screen display unit 420, a communication I/F unit 430, a storage unit 440, and an arithmetic processing unit 450 as main constituent elements thereof. Note that the agitation determination apparatus 400 is equipped with standard functions such as a clock function indicating time.

The operation input unit 410 is configured of an operation input device such as a keyboard and/or a mouse. The operation input unit 410 detects an operation input by the medical staff operating the agitation determination apparatus 400, and outputs it to the arithmetic processing unit 450.

The screen display unit 420 is configured of a screen display device such as an LCD (a Liquid Crystal Display). The screen display unit 420 can display various kinds of information stored in the storage unit 440, such as sensing data 442, connection condition information 443, score information 444, and result information 445, on the screen according to an instruction from the arithmetic processing unit 450. Note that the screen display unit 420 may be set up at a place remote from the place at which the arithmetic processing unit 450 and the like are set up. For example, only the screen display unit 420 among the configurations included in the agitation determination apparatus 400 may be set up at the nurse station. In this case, the arithmetic processing unit 450 and the like may be set up at a place different from the screen display unit 420, such as a server room.

The communication I/F unit 430 is configured of a data communication circuit. The communication I/F unit 430 carries out data communication between the agitation determination apparatus 400 and an external apparatus such as the bed terminal 300 connected via wireless communication or the like or a mobile terminal carried by the medical staff.

The storage unit 440 is a storage device such as a hard disk or a memory. FIG. 5 illustrates one example of information stored in the storage unit 440. As illustrated in FIG. 5, the storage unit 440 stores processing information required for various kinds of processing by the arithmetic processing unit 450, and a program 446 therein. The program 446 realizes the various kinds of processing units by being read in and executed by the arithmetic processing unit 450. The program 446 is read in from an external apparatus or a recording medium in advance via a data input/output function such as the communication IN unit 430, and is stored in the storage unit 440. Examples of main information stored in the storage unit 440 include an agitation determination-use model 441, the sensing data 442, the connection condition information 443, the score information 444, and the result information 445.

The agitation determination-use model 441 is a model for calculating the agitation determination-use score based on the vital data and/or the body motion data acquired by the sensor 210. For example, the agitation determination-use model 441 receives information according to the vital data and/or the body motion data as an input and outputs the agitation determination-use score. The agitation determination-use model 441 is, for example, a trained model generated in advance by carrying out machine learning using a support vector machine (SVM), a neural network, or the like in an external apparatus or the like. For example, the machine learning is carried out by using data generated by labeling previously measured vital data and/or body motion data with the presence or absence of the agitation as supervisory data. The agitation determination-use model 441 is acquired from the external apparatus or the like via the communication I/F unit 430 or the like, and is stored in the storage unit 440.

Note that the agitation determination-use model 441 may include a plurality of types of models according to the type of the data. For example, the agitation determination-use model 441 may include a vital model to which the vital data is input, and a body motion model to which the body motion data is input. Further, the agitation determination-use model 441 may include a plurality of models according to the type of the vital data and/or the body motion data, such as the acceleration and the vocal volume. The agitation determination-use model 441 may be a model different from the above-described examples.

Further, the agitation determination-use score output from the agitation determination-use model 441 is an index for determining whether the patient is agitated or whether the patient exhibits the predictive sign of agitation. The agitation determination-use score is, for example, a value from 0 to 1, inclusive. The agitation determination-use score indicates that the patient is agitated or exhibits the predictive sign of agitation as the value thereof is closer to 1, and indicates that the patient is not agitated or does not exhibit the predictive sign of agitation as the value thereof is closer to 0. The agitation determination-use score may be an index expressed using two values that are 1 indicating that the patient is agitated or exhibits the predictive sign of agitation and 0 indicating that the patient is not in the agitated state or does not exhibit the predictive sign of agitation. The agitation determination-use score may be an index indicating the intensity of the agitation, such as an intensity set to 2 for strong agitation and 1 for slight agitation.

Further, the data input to the agitation determination-use model 441 may be the vital data and/or the body motion data itself or themselves, or may be various kinds of feature amounts calculated by subjecting the time-series vital data and/or body motion data to processing for converting the time-series data into the feature amount such as averaging processing or differential processing. Further, the agitation determination-use model 441 may be configured to input one type of vital data and/or body motion data or may be configured to input a plurality of types of vital data and/or body motion data.

The sensing data 442 includes the data measured by the sensor 210. For example, the sensing data 442 stores therein the vital data, the body motion data, and/or the like acquired by the sensor 210 individually for each identification information of the sensor apparatus 200.

For example, FIG. 6 illustrates one example of time-series data of the heart rate, which is one type of the vital data. In the case of FIG. 6, an x axis represents time and a y axis represents the heart rate.

The connection condition information 443 indicates a connection condition between apparatuses present in a route taken until the agitation determination apparatus 400 acquires the data acquired by the sensor 210, such as a connection condition between the sensor apparatus 200 and the bed terminal 300 and a connection condition between the bed terminal 300 and the agitation determination apparatus 400. For example, the connection condition information 443 indicates whether a communicable connection is established between the sensor apparatus 200 and the bed terminal 300 and between the bed terminal 300 and the agitation determination apparatus 400. The connection condition information 443 is updated according to, for example, the information received from the bed terminal 300 and/or the connection condition between the agitation determination apparatus 400 and the bed terminal 300.

The score information 444 includes the agitation determination-use score, which is the index for determining whether the patient is agitated (or the patient exhibits the predictive sign of agitation). For example, the score information 444 includes the identification information of the sensor apparatus 200 and the agitation determination-use score in association with each other.

FIG. 7 illustrates one example of the agitation determination-use score calculated by the score calculation unit 455 based on the vital data illustrated in FIG. 6. In the case of FIG. 7, an x axis represents time and a y axis represents the agitation determination-use score. As illustrated in FIG. 7, the agitation determination-use score is expressed by, for example, a value from 0 to 1, inclusive. The agitation determination-use score indicates that the patient is agitated or exhibits the predictive sign of agitation as the value thereof is closer to 1, and indicates that the patient is not agitated or does not exhibit the predictive sign of agitation as the value thereof is closer to 0.

The result information 445 includes, for example, information indicating a result of the determination made by an agitation state determination unit 456 based on the score information 444. For example, the result information 545 includes the identification information of the sensor apparatus 200 and the information indicating the result of the agitation determination.

The arithmetic processing unit 450 includes a microprocessor such as an MPU and a peripheral circuit thereof. The arithmetic processing unit 450 reads in the program 446 from the storage unit 440 and executes it, thereby causing the above-described hardware and the program 446 to cooperate with each other to realize various kinds of processing units. Examples of main processing units realized by the arithmetic processing unit 550 include a data acquisition unit 451, an error detection unit 452, an error cause estimation unit 453, a correction instruction unit 454, the score calculation unit 455, the agitation state determination unit 456, and a notification unit 457.

The data acquisition unit 451 acquires the body motion data and/or the vital data, the identification information, and the like transmitted from the bed terminal 300 via the communication IN unit 430. Then, the data acquisition unit 451 stores the acquired body motion data and/or vital data into the storage unit 440 in association with the identification information as the sensing data 442.

The agitation determination system 100 makes the agitation determination using the sensing data. However, the agitation state cannot be correctly determined if an error has occurred for some reason. Therefore, the agitation determination system 100 includes the error detection unit 452, the error cause estimation unit 453, and the correction instruction unit 454, which will be described now, thereby allowing an appropriate action to be taken against the error that has occurred.

The error detection unit 452 detects the occurrence of an error using the data used to make the agitation determination. More specifically, the error detection unit 452 detects the occurrence of an error using the vital data (or the body motion data).

In the case of the present exemplary embodiment, the error detection unit 452 detects the occurrence of an error based on the acquisition condition of the vital data, the condition of the vital data, and/or the like. For example, the error detection unit 452 determines that an error has occurred when at least one of the pieces of vital data becomes unable to be acquired normally. Note that the error detection unit 452 can determine that the vital data becomes unable to be acquired normally using at least one of, for example, a time stamp indicating the time at which the data is acquired or the data itself. For example, the error detection unit 452 can determine whether the vital data is acquired normally by, for example, comparing the time stamp and the current time, comparing the time stamps of a plurality of pieces of vital data, comparing the time stamps of the vital data and the body motion data, or confirming the data.

More specifically, for example, the error detection unit 452 can determine whether the vital data is acquired normally based on a result of comparing the time stamp of the last vital data included in the sensing data 442 (the time at which the vital data is acquired last), and the time at which the detection is attempted. For example, the error detection unit 452 compares the time stamp of the last vital data included in the sensing data 442, and the time at which the detection is attempted. Then, if the difference between the time stamp and the time at which the detection is attempted exceeds a predetermined allowable range such as 30 seconds or shorter, the error detection unit 452 determines that the vital data is not acquired normally and detects the occurrence of an error.

Further, the error detection unit 452 can determine whether the vital data is acquired normally based on a result of confirming whether the vital data included in the sensing data 442 satisfies a predetermined criterion. For example, the error detection unit 452 confirms whether the vital data included in the sensing data 442 satisfies the predetermined criterion. Then, for example, the error detection unit 452 determines that the vital data is not acquired normally and detects the occurrence of an error based on the predetermined criterion, such as a criterion that 50% or more of pieces of vital data within a predetermined time indicates a value outside a predetermined range, the match between the time difference of the heart rate and the time of the heart rate is lower than 90%, or the number of pieces of data of the heart rate acquired within a predetermined time falls within 90% of the ideal number of pieces of data calculated from the heart rate.

The error detection unit 452 detects the occurrence of an error based on the time stamp and/or the measured value such as the vital data included in the sensing data 442 in this manner by way of example.

Note that the sensing data 442 may include a plurality of types of data as described above. More specifically, the sensing data 442 may, for example, include the vital data and the body motion data, or include a plurality of types of vital data and/or a plurality of types of body motion data. In the case where the sensing data 442 includes a plurality of types of data in this manner, the error detection unit 452 can detect the occurrence of an error if even any one of the plurality of types of data satisfies a criterion like the above-described examples.

When the error detection unit 453 detects an error, the error cause estimation unit 452 estimates the cause for this error. For example, the error cause estimation unit 453 estimates the cause for the error based on the acquisition condition of the vital data, the connection condition information 443, and/or the like. For example, the error cause estimation unit 453 estimates the cause for the error based on the time stamp of the vital data, the connection condition information 443, and/or the like. Note that the error cause estimation unit 453 may estimate a cause location as the error cause. More specifically, the error cause estimation unit 453 can identify the location where the error has occurred based on the state of the vital data and/or the body motion data included in the sensing data 442, the acquisition condition thereof, the connection condition information 443, and/or the like. Further, the error cause estimation unit 453 instructs the correction instruction unit 454 to perform processing according to the result of the estimation.

More specifically, for example, suppose that the error detection unit 452 detects an error according to the satisfaction of the vital data or the like included in the sensing data 442 with the predetermined criterion. In this case, the error cause estimation unit 453 estimates that the error is caused by, for example, a contact failure between the sensor 210 that acquires the data satisfying the predetermined criterion and the skin of the patient. Further, the error cause estimation unit 453 can identify the sensor 210 as the location where the error has occurred. In this case, the error cause estimation unit 453 can instruct the correction instruction unit 454 to notify an external apparatus or the like that the error such as the contact failure has occurred in the identified sensor 210 or the bed terminal 300.

On the other hand, suppose that the error detection unit 452 detects an error based on the result of comparing the time stamp and the time at which the detection is attempted. In this case, for example, if the difference between the time stamp and the time exceeds the allowable range with respect to only a part of the types among the plurality of types of data included in the sensing data 442, the error cause estimation unit 453 estimates that the error is caused by a contact failure raised on the sensor 210 acquiring the data with respect to which the difference between the time stamp and the time exceeds the allowable range, the patient not wearing the sensor 210, or the like. Further, the error cause estimation unit 453 can identify the sensor 210 as the location where the error has occurred. In this case, the error cause estimation unit 453 can instruct the correction instruction unit 454 to notify the external apparatus or the like that the error such as the contact failure or the patient not wearing the sensor 210 has occurred in the identified sensor 210 or the bed terminal 300.

On the other hand, if the difference between the time stamp and the time exceeds the allowable range with respect to all of the pieces of data included in the sensing data 442, the error cause estimation unit 453, for example, estimates the cause for the error and/or identifies the location where the error has occurred based on the connection condition information 443.

For example, if determining that the bed terminal 300 and the agitation determination apparatus 400 are not communicably connected based on the connection condition information 443, the error cause estimation unit 453 estimates that the error is caused by a power-off, a Wi-Fi error, or the like that has occurred in the bed terminal 300. Further, the error cause estimation unit 453 can identify the bed terminal 300 as the location where the error has occurred. In this case, the error cause estimation unit 453 can instruct the correction instruction unit 454 to notify the external apparatus or the like that the error has occurred in the bed terminal 300. Note that the error cause estimation unit 453 may be configured to also instruct the correction instruction unit 454 to instruct the bed terminal 300 to restart the communication function or restart the power source of the terminal itself along with the above-described notification.

On the other hand, for example, if determining that the sensor apparatus 200 and the bed terminal 300 are not connected while the bed terminal 300 and the agitation determination apparatus 400 are connected based on the connection condition information 443, the error cause estimation unit 453 estimates that the error is caused by the patient getting out of the bed, a power-off of the sensor apparatus 200, the occurrence of an error in the bed terminal 300, or the like. Further, the error cause estimation unit 453 can identify the sensor apparatus 200 or the bed terminal 300 as the location where the error has occurred. In this case, the error cause estimation unit 453 can instruct the correction instruction unit 454 to notify the external apparatus or the like that the patient may get out of the bed, that the error may have occurred in the sensor apparatus 200 or the bed terminal 300, or the like.

On the other hand, for example, if determining that the bed terminal 300 and the agitation determination apparatus 400 are connected and the sensor apparatus 200 and the bed terminal 300 are also connected based on the connection condition information 443, the error cause estimation unit 453 estimates that the error is caused by the occurrence of an error in the bed terminal 300, detachment of all of the sensors 210, or the like. Further, the error cause estimation unit 453 can identify the sensor apparatus 200 including the sensor 210 or the bed terminal 300 as the location where the error has occurred. In this case, the error cause estimation unit 453 can instruct the correction instruction unit 454 to instruct the bed terminal 300 to restart the power source of the terminal itself. Further, the error cause estimation unit 453 can instruct the correction instruction unit 454 to notify the external apparatus or the like that the sensor 210 may be detached from the patient or the like along with the above-described restart instruction.

Note that the error cause estimation unit 453 may instruct the correction instruction unit 454 to instruct the agitation determination apparatus 400 to restart itself if determining that, for example, the agitation determination apparatus 400 itself is frozen.

The error cause estimation unit 453 estimates the cause for the error based on the state of the vital data and/or the body motion data included in the sensing data 442, the acquisition condition thereof, the connection condition information 443, and/or the like in this manner by way of example. Further, the error cause estimation unit 453 can identify the location where the error has occurred. Then, the error cause estimation unit 453 instructs the correction instruction unit 454 to perform processing according to the result of the estimation and the identification.

Note that the estimation processing performed by the error cause estimation unit 453 is not limited to the above-described examples. The error cause estimation unit 453 may, for example, estimate the cause for the error and/or identify the location where the error has occurred by a method different from the above-described examples. Further, the error cause estimation unit 453 may instruct each processing unit to perform processing according to a specific result different from the above-described examples. Further, for example, the error cause estimation unit 453 can also be configured to acquire information indicating, for example, an operation condition of a sensor application that performs measurement processing from the sensor apparatus 200 or the bed terminal 300. In this case, the error cause estimation unit 453 may, for example, estimate the cause for the error and/or identify the location where the error has occurred based on the acquired information.

The correction instruction unit 454 issues a correction instruction corresponding to the cause for the error estimated by the error cause estimation unit 453. The correction instruction unit 454, for example, notifies the external apparatus or the like and/or issues the restart instruction directed to the identified location where the error has occurred as the correction instruction.

Note that the external apparatus or the like may be notified of the correction content and/or the like or may be notified of only the occurrence of the error in the notification to the external apparatus or the like. The correction instruction unit 454 may notify the external apparatus or the like of candidates for the correction content when, for example, the correction content cannot be estimated in a pinpointed manner. Further, the correction instruction unit 454 can notify the external apparatus or the like of the occurrence location along therewith when the occurrence location is identified by the error cause estimation unit 453. Further, the correction instruction unit 454 may control the notification destination according to the error type or the like. In the case of, for example, the contact failure of the sensor 210, the correction instruction unit 454 notifies the mobile terminal carried by medical staff in charge thereof. On the other hand, in the case of such a system-related error that the apparatus should be restarted, the correction instruction unit 454 notifies, for example, an administrator apparatus that administers the system. The correction instruction unit 454 may control the notification destination according to the error type or the like in this manner by way of example.

Further, the correction instruction unit 454 can transmit the restart instruction to the sensor apparatus 200 or the bed terminal 300 according to the instruction from the error cause estimation unit 453. More specifically, for example, the correction instruction unit 454 receives the instruction to transmit the restart instruction from the error cause estimation unit 453. In response thereto, the correction instruction unit 454 transmits the restart instruction instructing the apparatus to restart itself to the sensor apparatus 200 or the bed terminal 300 specified in the instruction to transmit the restart instruction.

The score calculation unit 455 and the agitation state determination unit 456 perform processing for determining the agitation state based on the sensing data 442. For example, the score calculation unit 455 and the agitation state determination unit 456 perform the processing for determining the agitation state while no error is detected by the error detection unit 452. For example, if normal data is included in the sensing data 442 although an error is detected by the error detection unit 452, the score calculation unit 455 and the agitation state determination unit 456 may continue determining the agitation state based on the normal data included in the sensing data 442. In the following description, an example of the processing performed by the score calculation unit 455 and the agitation state determination unit 456 will be described. Note that the processing for making the agitation determination is not limited thereto.

The score calculation unit 455 calculates the agitation determination-use score using the agitation determination-use model 441.

For example, the score calculation unit 455 acquires the vital data including the time-series data of the heart rate like the example illustrated in FIG. 6 by referring to the sensing data 442. Further, the score calculation unit 455 inputs the acquired data to the agitation determination-use model 441, and calculates the agitation determination-use score at each time like the example illustrated in FIG. 7. After that, the score calculation unit 455 stores information indicating the calculated agitation determination-use score into the storage unit 440 as the score information 444.

Note that the score calculation unit 455 may input the time-series data itself to the agitation determination-use model 441, or may input various kinds of feature amounts calculated by subjecting the time-series data to processing for converting the time-series data into the feature amount such as averaging processing or differential processing to the agitation determination-use model 441.

The agitation state determination unit 456 determines the agitation state of the patient based on the agitation determination-use score included in the score information 444. For example, the agitation state determination unit 456 determines whether the patient is agitated or whether the patient exhibits the predictive sign of agitation as the agitation state of the patient. Then, the agitation state determination unit 456 stores the result of the determination into the storage unit 440 as the result information 445. For example, the agitation state determination unit 456 stores information indicating such a result that the patient is determined to be agitated into the storage unit 440 as the result information 445.

For example, the agitation state determination unit 456 includes a determination threshold value to be compared with the agitation determination-use score in advance. Then, the agitation state determination unit 456 makes the determination based on the agitation determination-use score and the agitation determination threshold value. For example, the agitation state determination unit 456 determines that the patient is agitated or the patient exhibits the predictive sign of agitation if the agitation determination-use score is equal to or higher than the agitation determination threshold value. On the other hand, the agitation state determination unit 456 determines that the patient is not agitated or the patient does not exhibit the predictive sign of agitation if the agitation determination-use score is lower than the agitation determination threshold value.

More specifically, for example, in the case of the agitation determination-use score illustrated in FIG. 8, the agitation determination-use score becomes equal to or higher than the determination threshold value during a period from 22:30 to slightly before 1:00 and slightly before 2:00 as illustrated in FIG. 9. Accordingly, the agitation state determination unit 456 determines that the patient is agitated during these times.

Note that FIG. 9 illustrates an example in which the determination threshold value is set to 0.5. However, the determination threshold value may be a value different from 0.5. The value of the determination threshold value may be set in any manner. The determination threshold value may be determined as appropriate according to, for example, attribute information of the patient, which will be described below.

The notification unit 457 provides a required output when the agitation state determination unit 554 determines that the patient is agitated.

For example, when the agitation state determination unit 554 determines that the patient is agitated, the notification unit 457 displays that the patient is in the agitated state or the like on the screen display unit 420 together with the identification information of the sensor apparatus 200. Further or alternatively, the notification unit 457 transmits the determination that the patient is in the agitated state or the like and the identification information of the sensor apparatus 200 to an external apparatus such as the bed terminal 300 relating to this patient or the mobile terminal carried by the medical staff in charge of this patient. The notification unit 457 may issue a notification in a manner different from the above-described example, such as lighting a lamp at an entrance of a room in which the patient is hospitalized.

Note that the agitation determination apparatus 400 may be configured to be able to spot medical staff (a mobile terminal) staying at a position closest to the location where the error has occurred or the patient in the agitated state based on, for example, the positional information of the mobile terminal. In the case where the agitation determination apparatus 400 is configured in this manner, the notification unit 457 may notify the mobile terminal carried by the spotted medical staff in the above-described manner.

This is an example of the configuration of the agitation determination system 100. Subsequently, examples of operations of the agitation determination apparatus 400 will be described with reference to FIG. 9. Note that the order of the operations of the agitation determination apparatus 400 illustrated in FIG. 9 is one example, and the operations of the agitation determination apparatus 400 are not limited thereto.

FIG. 9 is a flowchart illustrating the examples of the operations of the agitation determination apparatus 400. Referring to FIG. 9, the data acquisition unit 451 acquires the body motion data and/or the vital data, the identification information of the server apparatus 200, and the like via the communication I/F unit 430 (step S101).

The error detection unit 452 detects an error that has occurred in the agitation determination system 100 based on, for example, the vital data included in the sensing data 442 (step S102). For example, the error detection unit 452 detects the occurrence of an error based on the result of the comparison between the time stamp and the time at which the detection is attempted, the state of the data included in the sensing data 442, and/or the like.

If the error detection unit 452 detects the occurrence of an error (YES in step S102), the error cause estimation unit 453 identifies the location where the error has occurred (step S103). For example, the error cause estimation unit 453 identifies the location where the error has occurred based on the state of the vital data and/or the body motion data included in the sensing data 442, the acquisition condition thereof, the connection condition information 443, and/or the like.

Further, if determining that the error may be able to be corrected by a restart based on the result of the identification (YES in step S104), the error cause estimation unit 453 instructs the correction instruction unit 454 to issue the restart instruction. In response thereto, the correction instruction unit 454 issues the restart instruction to the sensor apparatus 200 or the bed terminal 300 (step S105). Note that the predetermined notification may be issued along with the restart instruction by the correction instruction unit 454. On the other hand, if determining that the correction by a restart is difficult (NO in step S104), the error cause estimation unit 453 instructs the correction instruction unit 454 to notify the external apparatus or the like of the occurrence of the error. In response thereto, the correction instruction unit 454 notifies the mobile terminal carried by the medical staff or the like of the occurrence of the error (step S106).

On the other hand, if the error detection unit 452 does not detect the occurrence of an error (NO in step S102), the agitation determination apparatus 400 makes the agitation determination (step S107). The agitation determination by the agitation determination apparatus 400 is made using, for example, the score calculation unit 455 and the agitation state determination unit 456. Note that the agitation determination by the agitation determination apparatus 400 is not limited to the described example.

These are examples of the operations of the agitation determination apparatus 400.

In this manner, the agitation determination apparatus 400 includes the error detection unit 452, the error cause estimation unit 453, and the correction instruction unit 454. According to such a configuration, when the error detection unit 452 detects the occurrence of an error, the error cause estimation unit 453 can identify the location where the error has occurred. As a result, the agitation determination apparatus 400 can, for example, issue the restart instruction and/or transmit the predetermined notification by the correction instruction unit 454 according to the result of the identification by the error cause estimation unit 453. This allows an appropriate action to be promptly taken even when, for example, the agitation determination system 100 falls into such a situation that the agitation state cannot be determined due to an error.

Note that the configuration of the agitation determination apparatus 400 is not limited to the example described with reference to FIG. 4. For example, FIG. 10 illustrates another example of the configuration of the agitation determination apparatus 400. Referring to FIG. 10, the arithmetic processing unit 450 of the agitation determination apparatus 400 can include, for example, a priority assignment unit 458.

The priority assignment unit 458 assigns a priority of the notification according to the result of the determination about the agitation state, such as the agitation determination-use score indicated by the score information 444. The notification unit 457 can, for example, issue the notification based on a notification content and/or a notification method according to the result of the priority assignment set by the priority assignment unit 458.

Now, the priority refers to an index indicating, for example, the importance, the necessity, or the urgency of the notification. The priority indicates that the notification has higher importance, necessity, urgency, or the like as the priority increases. For example, when it is necessary to make the medical staff or the like aware of the occurrence of the error immediately, a high priority is assigned assuming that the notification of this error has high importance, necessity, urgency, or the like.

For example, the priority assignment unit 458 can assign the priority based on a previous agitation determination-use score such as scores acquired until yesterday, one hour ago, or the like. For example, the priority assignment unit 458 refers to the previous agitation determination-use score. Then, the priority assignment unit 458 assigns the priority according to the previous agitation score. For example, the priority assignment unit 458 determines that the priority is higher as the previous agitation determination-use score is higher. The priority assignment unit 458 may assign the priority based on whether the previous agitation determination-use score exceeds a predetermined previous threshold value (may be any value). For example, if the previous agitation determination-use score exceeds the previous threshold value, the priority assignment unit 458 determines the priority is high. In this case, the correction instruction unit 454 can issue a further intense notification indicating the occurrence of the error. For example, the correction instruction unit 454 realizes the further intense notification by employing at least one method among outputting a notification sound, turning up the notification sound, changing the notification sound, notifying a plurality of destinations, and the like. On the other hand, if the previous agitation determination-use score is equal to or lower than the previous threshold value, the priority assignment unit 458 determines that the priority is low. In this case, the correction instruction unit 454 can issue a soft notification indicating the error. For example, the correction instruction unit 454 realizes the soft notification by employing at least one method among refraining from outputting the sound, turning down the sound, and the like. Note that the priority assignment unit 458 can determine that the priority is intermediate (or refrain from determining the priority) if the previous agitation score is absent. In this case, the correction instruction unit 454 may employ an intermediate method between the intense notification and the soft notification, such as outputting a quiet sound. Further, the previous threshold value may be one or may include a plurality of different values. In the case where the previous threshold value includes a plurality of values, the priority assignment unit 458 can determine a stepwise priority. By issuing the notification with the priority reflected therein in this manner, the agitation determination apparatus 400 can quickly make the medical staff or the like receiving the notification aware of the importance or the like of the notification. Then, this allows the medical staff or the like to further promptly take an appropriate action against the occurrence of the error.

Further, FIG. 11 illustrates another example of the configuration of the agitation determination apparatus 400. Referring to FIG. 11, the arithmetic processing unit 450 of the agitation determination apparatus 400 can include, for example, attribute information acquisition unit 459 in addition to or instead of the priority assignment unit 458.

The attribute information acquisition unit 459 acquires the attribute information of the patient, which is the target. For example, the attribute information acquisition unit 459 acquires medical record information of the target from an external apparatus or the like, and acquires attribute information such as an age, a gender, and a paralysis state.

The attribute information acquired by the attribute information acquisition unit 459 can be utilized, for example, when the determination threshold value is adjusted. Further, the attribute information acquired by the attribute information acquisition unit 459 can be utilized, for example, when the priority assignment unit 458 determines the priority. For example, the priority assignment unit 458 determines whether the occurrence of the agitation is highly likely based on the prescription, the optional comment field, the name of the disease, the age, the gender, and the like indicated by the attribute information. Then, the priority assignment unit 458 determines that the priority is high if determining that the occurrence of the agitation is highly likely. On the other hand, the priority assignment unit 458 determines that the priority is low if determining that the occurrence of the agitation is less likely. The priority assignment unit 458 may determine the notification method based on the attribute information in this manner by way of example. Note that the priority assignment unit 458 may determine the priority based on a combination of the previous agitation determination-use score and the attribute information.

Further, the agitation determination system 100 includes the plurality of sensor apparatuses 200, the plurality of bed terminals 300, and the like as described above. Therefore, errors may occur at a plurality of locations around the same time as illustrated in FIG. 12.

When errors have occurred at a plurality of locations around the same time in this manner, the correction instruction unit 454 can, for example, adjust the order in which the mobile terminal carried by the medical staff is notified and/or generate information indicating the order of the correction and add it in the notification according to the priority determined by the priority assignment unit 458. In other words, when errors have occurred at a plurality of locations around the same time, the priority assignment unit 458 can assign priorities to notifications corresponding to this plurality of locations. Further, the correction instruction unit 454 determines, for example, the notification content and/or the notification method according to the priority determined by the priority assignment unit 458. For example, the correction instruction unit 454 can issue the notification based on the notification content and/or the notification method according to the priority determined by the priority assignment unit 458. Note that the priority assignment unit 458 may determine the priority based on the descending order of the previous agitation determination-use score (an average value, a largest value, or the like), the descending order of the likelihood that the agitation occurs, which is determined based on the attribute information, and/or the like, similarly to the above-described example. Note that the priority in this case may be information indicating the order of the notification. Further, the correction instruction unit 454 can generate, for example, the information indicating the order of the correction and add it in the notification.

Note that FIGS. 4, 10, and 11 illustrate the example in which the functions as the agitation determination apparatus 400 are realized by one information processing apparatus. However, the functions as the agitation determination apparatus 400 may be realized by, for example, a plurality of information processing apparatuses connected via a network.

### Second Exemplary Embodiment

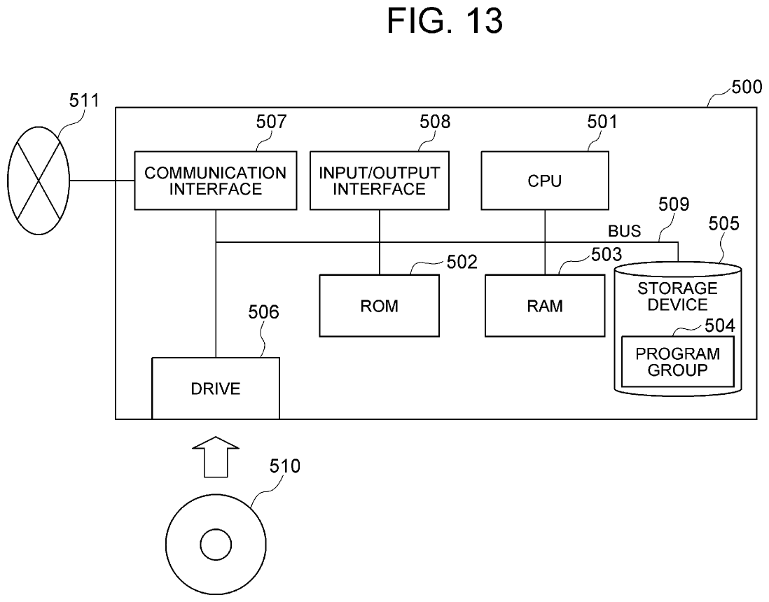

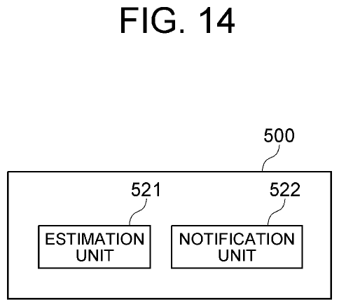

Next, a second exemplary embodiment of the present disclosure will be described with reference to FIGS. 13 and 14. The second exemplary embodiment will be described regarding an overview of the configuration of a determination apparatus 500.

FIG. 13 illustrates an example of the hardware configuration of the determination apparatus 500. Referring to FIG. 13, the determination apparatus 500 has the following hardware configuration as one example.
Central Processing Unit (CPU) 501 (arithmetic unit)
Read Only Memory (ROM) 502 (storage unit)
Random Access Memory (RAM) 503 (storage unit)
Program group 504 to be loaded to the RAM 503
Storage device 505 storing therein the program group 504
Drive 506 that performs reading and writing on a recording medium 510 outside the information processing apparatus
Communication interface 507 connecting to a communication network 511 outside the information processing apparatus
Input/output interface 508 for performing input/output of data
Bus 509 connecting the constituent elements

Further, the determination apparatus 500 can realize functions as an estimation unit 521 and a notification unit 522 illustrated in FIG. 14 through acquisition of the program group 504 by the CPU 501 and execution thereof by this CPU 501. Note that the program group 504 is, for example, stored in the storage device 505 or the ROM 502 in advance, and is loaded to the RAM 503 or the like and executed by the CPU 501 as needed. Alternatively, the program group 504 may be provided to the CPU 501 via the communication network 511, or may be stored in the recording medium 510 in advance and read out by the drive 506 and provided to the CPU 501.

Note that FIG. 13 illustrates an example of the hardware configuration of the determination apparatus 500. The hardware configuration of the determination apparatus 500 is not limited to the above-described example. For example, the determination apparatus 500 may be configured of a part of the above-described configuration, such as not including the drive 506.

The estimation unit 521 estimates a cause for an error based on data used when an agitation state is determined.

The notification unit 522 issues a notification according to a result of the estimation by the estimation unit 521.

In this manner, the determination apparatus 500 includes the estimation unit 521 and the notification unit 522. According to such a configuration, the notification unit 522 can issue a notification according to the result of the estimation by the estimation unit 521 based on the data used when the agitation state is determined. As a result, the determination apparatus 500 allows an appropriate action to be promptly taken even when falling into such a situation that the agitation state cannot be determined due to an error.

Note that the above-described determination apparatus 500 can be realized by incorporating a predetermined program into this determination apparatus 500. More specifically, a program according to another exemplary embodiment of the present invention is a program for causing the determination apparatus 500 to realize the estimation unit 521, which estimates the cause for the error based on the data used when the agitation state is determined, and the notification unit 522, which issues the notification according to the result of the identification by the estimation unit 521.

Further, a notification method realized by the above-described determination apparatus 500 is a method for causing a determination apparatus to estimate a cause for an error based on data used when an agitation state is determined and issue a notification according to a result of the estimation.

Even the invention of the program (or a recording medium) or the notification method configured in the above-described manner can also bring about advantageous effects equivalent to the above-described determination apparatus 500, thereby achieving the above-described object of the present invention. Note that the present invention may be applied to another apparatus that determines an error based on measurement data of a target.

### <Supplementary Notes>

The whole or part of the exemplary embodiments disclosed above can be described as, but not limited to, the following supplementary notes. Hereinafter, outlines of the determination apparatus and the like according to the present invention will be described. However, the present invention is not limited to the configurations described below.

### (Supplementary Note 1)

A determination apparatus comprising:

an estimation unit configured to estimate a cause for an error based on data used when an agitation state is determined; and

a notification unit configured to issue a notification according to a result of the estimation by the estimation unit.

### (Supplementary Note 2)

The determination apparatus according to supplementary note 1, wherein

the estimation unit estimates the cause for the error based on a state of the data used when the agitation state is determined.

### (Supplementary Note 3)

The determination apparatus according to supplementary note 1 or 2, wherein

in a case where the data used when the agitation state is determined is in a state of satisfying a predetermined criterion, the estimation unit estimates that the cause for the error is present in a sensor configured to acquire the data satisfying the criterion.

### (Supplementary Note 4)

The determination apparatus according to any one of supplementary notes 1 to 3, wherein

the estimation unit estimates the cause for the error based on an acquisition condition of the data used when the agitation state is determined.

### (Supplementary Note 5)

The determination apparatus according to any one of supplementary notes 1 to 4, wherein

the estimation unit estimates the cause for the error based on information indicating a time of acquisition of the data used when the agitation state is determined.

### (Supplementary Note 6)

The determination apparatus according to any one of supplementary notes 1 to 5, wherein

the estimation unit estimates the cause for the error based on information indicating a connection condition between apparatuses present between the sensor configured to acquire the data used when the agitation state is determined and the determination apparatus.

### (Supplementary Note 7)

The determination apparatus according to any one of supplementary notes 1 to 6, further comprising a correction instruction unit configured to issue a restart instruction according to the result of the estimation by the estimation unit.

### (Supplementary Note 8)

The determination apparatus according to any one of supplementary notes 1 to 7, further comprising:

a priority assignment unit configured to determine a priority serving as an index indicating importance of the notification according to information indicating a result of the determination about agitation determined based on the data used when the agitation state is determined, wherein

the notification unit issues the notification according to the priority determined by the priority assignment unit.

### (Supplementary Note 9)

The determination apparatus according to supplementary note 8, wherein

the priority assignment unit determines the priority based on attribute information indicating an attribute of a target.

### (Supplementary Note 10)

The determination apparatus according to any one of supplementary notes 1 to 9, further comprising:

a detection unit configured to detect the occurrence of the error based on the data used when the agitation state is determined, wherein

the estimation unit estimates the cause for the error according to the detection of the occurrence of the error by the detection unit.

### (Supplementary Note 11)

A notification method for causing a computer to:

estimate a cause for an error based on data used when an agitation state is determined; and

issue a notification according to a result of the estimation.

### (Supplementary Note 12)

A recording medium recording a program for causing a computer to realize processing including:

estimating a cause for an error based on data used when an agitation state is determined; and

issuing a notification according to a result of the estimating.

Note that the program disclosed in each of the above-described exemplary embodiments and the supplementary notes is, for example, stored in a storage device or recorded in a computer-readable recording medium. For example, the recording medium is a portable medium such as a flexible disk, an optical disk, a magneto-optical disk, or a semiconductor memory.

While the present invention has been described with reference to the exemplary embodiments described above, the present invention is not limited to the above-described exemplary embodiments. The form and details of the present invention can be changed within the scope of the present invention in various manners that can be understood by those skilled in the art.

### REFERENCE SIGNS LIST

100 agitation determination system
200 sensor apparatus
210 sensor
220 transmission/reception unit
300 bed terminal
310 transmission/reception unit
320 screen display unit
400 agitation determination apparatus
410 operation input unit
420 screen display unit
430 communication I/F unit
440 storage unit
441 agitation determination-use model
442 sensing data
443 connection condition information
444 score information
445 result information
446 program
450 arithmetic processing unit
451 data acquisition unit
452 error detection unit
453 error cause estimation unit
454 correction instruction unit
455 score calculation unit
456 agitation state determination unit
457 notification unit
458 priority assignment unit
459 attribute information acquisition unit
500 determination apparatus
501 CPU
502 ROM
503 RAM
504 program group
505 storage device
506 drive
507 communication interface
508 input/output interface
509 bus
510 recording medium
511 communication network
521 estimation unit
522 notification unit

What is claimed is:

1. A determination apparatus comprising:
at least one memory configured to store processing instructions; and
at least one processor configured to execute the processing instructions to:
estimate a cause for an error based on data used when an agitation state is determined; and
issue a notification according to a result of the estimation.

2. The determination apparatus according to claim 1, wherein the at least one processor is configured to execute the processing instructions to
estimate the cause for the error is estimated based on a state of the data used when the agitation state is determined.

3. The determination apparatus according to claim 1, wherein the at least one processor is configured to execute the processing instructions to,
in a case where the data used when the agitation state is determined is in a state of satisfying a predetermined criterion, estimate that the cause for the error is present in a sensor configured to acquire the data satisfying the criterion.

4. The determination apparatus according to claim 1, wherein the at least one processor is configured to execute the processing instructions to
estimate the cause for the error based on an acquisition condition of the data used when the agitation state is determined.

5. The determination apparatus according to claim 1, wherein the at least one processor is configured to execute the processing instructions to
estimate the cause for the error based on information indicating a time of acquisition of the data used when the agitation state is determined.

6. The determination apparatus according to claim 1, wherein the at least one processor is configured to execute the processing instructions to
estimate the cause for the error based on information indicating a connection condition between apparatuses present between the sensor configured to acquire the data used when the agitation state is determined and the determination apparatus.

7. The determination apparatus according to claim 1, wherein the at least one processor is configured to execute the processing instructions to
issue a restart instruction according to the result of the estimation by the estimation unit.

8. The determination apparatus according to claim 1, wherein the at least one processor is configured to execute the processing instructions to:
determine a priority serving as an index indicating importance of the notification according to information indicating a result of the determination about agitation determined based on the data used when the agitation state is determined; and
issue the notification according to the determined priority.

9. The determination apparatus according to claim 8, wherein the at least one processor is configured to execute the processing instructions to
determine the priority based on attribute information indicating an attribute of a target.

10. The determination apparatus according to claim 1, wherein the at least one processor is configured to execute the processing instructions to:
detect the occurrence of the error based on the data used when the agitation state is determined; and
estimate the cause for the error according to the detection of the occurrence of the error.

11. A notification method for causing a computer to:
estimate a cause for an error based on data used when an agitation state is determined; and
issue a notification according to a result of the estimation.

12. A non-transitory computer readable recording medium recording a program for causing a computer to realize processing including:
estimating a cause for an error based on data used when an agitation state is determined; and
issuing a notification according to a result of the estimating.
